(19) United States
(12) Patent Application Publication
(10) Pub. No.: US 2022/0409280 A1
(43) Pub. Date: Dec. 29, 2022

(54) CUSTOMIZED GUIDE, AND APPARATUS AND METHOD FOR MANUFACTURING SAME

(71) Applicants: UNIVERSITY OF ULSAN FOUNDATION FOR INDUSTRY COOPERATION, Ulsan (KR); THE ASAN FOUNDATION, Seoul (KR)

(72) Inventors: Joon Bum KIM, Seoul (KR); Dong Hyun YANG, Seoul (KR); Namkug KIM, Seoul (KR); Taehun KIM, Seoul (KR)

(21) Appl. No.: 17/777,203

(22) PCT Filed: Nov. 13, 2020
(86) PCT No.: PCT/KR2020/016024

§ 371 Date: May 16, 2022

(30) Foreign Application Priority Data
Nov. 15, 2019 (KR) 10-2019-0146793

(51) Int. Cl. A61B 34/10

(52) U.S. Cl. CPC A61B 34/10; A61B 2034/108; A61F 2/064

(57) ABSTRACT

A customized guide includes a body having a shape corresponding to a main vessel in biological tissues that includes the main vessel and an accessory vessel branching from the main vessel, and an indicator provided in the body to indicate a branching location of the accessory vessel.

### TECHNICAL FIELD

The present disclosure relates to a customized guide, a manufacturing apparatus and method thereof, and more particularly, to a guide manufactured according to a patient's anatomical structure and an apparatus and method for manufacturing the guide.

### BACKGROUND ART

Open surgery repair of a thoracoabdominal aorta requires a graft for aortic reconstruction, which includes a thoracoabdominal graft corresponding to the aorta of biological tissues and a vascular graft corresponding to the arterioles branching from the aorta, and the thoracoabdominal graft and the vascular graft are manufactured in various sizes in advance.

According to a related art, prior to open surgery repair of thoracoabdominal aorta, a patient's medical image is analyzed to identify a location of the aorta and arteriole, a direction of the vascular graft is calculated by adding an angle of the arterioles, that is, the visceral artery and the intercostal artery, at the center of the aorta in a cross section perpendicular to a blood flow direction of the aorta, and a graft for aortic reconstruction is manufactured by connecting the vascular graft to the thoracoabdominal graft according to the calculated direction.

However, since the angle is calculated considering only the direction of the arterioles, there was a limitation in reconstructing the vascular graft at an accurate location.

### RELATED ART DOCUMENT

(Patent Document 1) Japanese Patent No. 5286352 (Registration Date: Jun. 07, 2013)

### DETAILED DESCRIPTION OF THE INVENTION

### Technical Problem

The present disclosure provides a customized guide in which a location of an accessory vessel branching from a main vessel is indicated according to a patient's anatomical structure, and a manufacturing apparatus and method for the guide.

The objects of the present disclosure are not limited to the above descriptions, and other objects that are not mentioned will be apparently understood by those skilled in the art from the following descriptions.

### Technical Solution to the Problem

In accordance with a first aspect of the present disclosure, there is provided a customized guide including: a body having a shape corresponding to a main vessel in biological tissues that includes the main vessel and an accessory vessel branching from the main vessel; an indicator provided in the body to indicate a branching location of the accessory vessel.

In accordance with a second aspect of the present disclosure, there is provided a customized guide manufacturing method performed in a customized guide manufacturing apparatus, the method including: generating three-dimensional model data for a target part including a main vessel in biological tissues and an accessory vessel branching from the main vessel; and generating a guide including an indicator that indicates a branching location of the accessory vessel in a body having a shape corresponding to the main vessel based on the three-dimensional model data.

In accordance with a third aspect of the present disclosure, there is provided a computer-readable non-transitory recording medium with a computer program stored therein, wherein the computer program, when executed by a processor, includes a command to implement a method which includes: generating three-dimensional model data for a target part including a main vessel in biological tissues and an accessory vessel branching from the main vessel; and generating a guide provided with an indicator that indicates a branching location of the accessory vessel in the body having a shape corresponding to the main vessel based on the three-dimensional model data.

In accordance with a fourth aspect of the present disclosure, there is provided a custom guide manufacturing apparatus including: a model generator configured to generate three-dimensional model data on a target part including a main vessel in biological tissues and an accessory vessel branching from the main vessel; and a guide generator configured to generate a guide including an indicator that indicates a branch location of the accessory vessel on the body having a shape corresponding to the main vessel based on the three-dimensional model data.

### Advantageous Effects of the Invention

According to an embodiment of the present disclosure, it is possible to provide a customized guide, in which the location of the accessory vessel branching from the main vessel is indicated according to the patient's anatomical structure, and also indicate the branching direction, and therefore, a location for a vascular graft to be installed in an thoracoabdominal graft and a direction of installation of the vascular graft may be guided when a graft for aortic reconstruction is manufactured.

In a method of using a customized guide according to an embodiment, it is possible to fix and couple the customized guide to the thoracoabdominal graft, and in this case, a corresponding location of the thoracoabdominal graft is changed to a convex shape by the indicator protruding from the body of the customized guide, and thus, it is possible to guide the corresponding location as a location of the vascular graft to be provided in the thoracoabdominal graft.

### BRIEF DESCRIPTION OF THE DRAWINGS

FIG. 1 is a diagram illustrating a configuration of a customized guide according to an embodiment of the present disclosure.

FIG. 2 is a block diagram of a customized guide manufacturing method according to the embodiment of the present disclosure.

FIG. 3 is an exemplary view of a contrasted tomography image that can be used for manufacturing a customized guide according to the embodiment of the present disclosure.

FIG. 4 is an exemplary diagram of 3D model data generated during a manufacturing process of a customized guide according to the embodiment of the present disclosure.

FIG. 5 is a flowchart illustrating a customized guide manufacturing method, which is performed in a customized guide manufacturing apparatus according to the embodiment of the present disclosure.

### BEST MODE FOR CARRYING OUT THE INVENTION

Advantages and features of the present disclosure, and a method of achieving them will become apparent with reference to an embodiment described later together with the accompanying drawings. However, the present disclosure is not limited to an embodiment disclosed below, but may be implemented in a variety of different forms. That is, the embodiment is provided to ensure that descriptions of the present disclosure are complete and to fully inform a scope of the invention to a person with ordinary knowledge in a technical field to which the present disclosure belongs, and the invention is only defined by the scope of claims.

In describing the embodiments of the present disclosure, if it is determined that detailed description of related known components or functions unnecessarily obscures the gist of the present disclosure, the detailed description thereof will be omitted. Further, the terminologies to be described below are defined in consideration of functions of the embodiments of the present disclosure and may vary depending on a user's or an operator's intention or practice. Accordingly, the definition thereof may be made on a basis of the content throughout the specification.

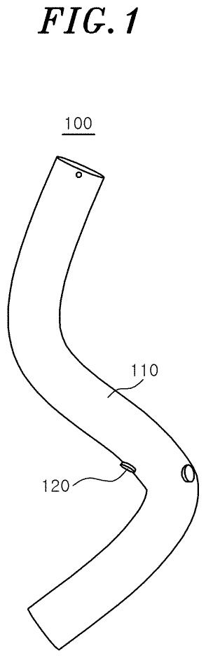

FIG. 1 is a diagram illustrating a configuration of a customized guide according to an embodiment of the present disclosure.

As shown in FIG. 1, a customized guide 100 includes a body 110 having a shape corresponding to a main vessel in biological tissues, which includes the main vessel and an accessory vessel branching from the main vessel. In addition, the customized guide 100 further includes an indicator 120 provided in the body 110 to indicate a branching location of the accessory vessel. Also, the customized guide 100 may further include a fixing hole 130 for fixing a thoracoabdominal graft and the customized guide 100.

Here, the indicator 120 may indicate a branching direction of the accessory vessel on the body 110 together with the branching location. For example, the indicator 120 may protrude from the body 110 to have a shape corresponding to a cross section perpendicular to a blood flow direction of the accessory vessel. In addition, the cross-section may be a cross-section of a portion connected to the body 110 in the accessory vessel. In a more detailed example, the cross-section of the accessory vessel may have a ring shape, and the indicator 120 may also have a ring shape.

Meanwhile, in the customized guide 100, an outer diameter of the body 110 may be identical to an inner diameter of the main vessel, or a difference between the outer diameter of the body 110 and the inner diameter of the main vessel may be smaller than a membrane thickness of the main vessel. For example, the outer diameter of the body 110 may be smaller than the inner diameter of the main vessel by an intima thickness of the main vessel. This is because, in the case of fitting the customized guide 100 to the thoracoabdominal graft, the fitting and coupling may be easily done by sliding between the customized guide 100 and the thoracoabdominal graft when the outer diameter of the body 110 is smaller than an inner diameter of the thoracoabdominal graft.

In the method of using the customized guide 100 according to the embodiment, when manufacturing a graft for aortic reconstruction prior to the open surgery repair of thoracoabdominal aorta, the graft is selected to fit a patient's aorta size from among pre-manufactured thoracoabdominal grafts of various sizes, the body 110 of the customized guide 100 is designed and manufactured to fit an outer diameter of the graft, and the body 110 of the customized guide 100 is fixed and coupled to the thoracoabdominal graft through the fixing hole 130 by using forceps.

Then, a corresponding location of the thoracicabdominal graft is changed to a convex shape by the indicator 120 protruding from the body 110 of the customized guide 100, and such a location changed to the convex shape is a location where to install the vascular graft. This means that, in manufacturing the graft for aortic reconstruction, the customized guide 100 guides a location where the vascular graft is to be provided. Here, since the indicator 120 indicates information on the branching direction through the directionality of the convex shape, it is also possible to guide the branching direction of the vascular graft to be installed at the corresponding location.

Accordingly, a worker who manufactures the graft for aortic reconstruction can easily install the vascular graft at an accurate location and in an accurate direction as guided by the customized guide 100.

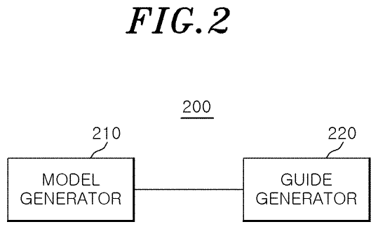

FIG. 2 is a block diagram of a customized guide manufacturing apparatus according to the embodiment of the present disclosure.

As shown in FIG. 2, a customized guide manufacturing apparatus 200 includes a model generator 210 and a guide generator 220.

The model generator 210 generates three-dimensional (3D) model data by dividing a target body part including a main vessel in biological tissues and an accessory vessel branching from the main vessel based on a medical image. For example, the biological tissues may include an aorta including an aortic dissection and an aortic aneurysm as a main vessel, and may include major vessels around the aorta, such as renal arteries, mesenteric arteries, celiac arteries, intercostal arteries, iliac arteries, and the like as accessory vessels. The model generator 210 may include a computing means such as a microprocessor.

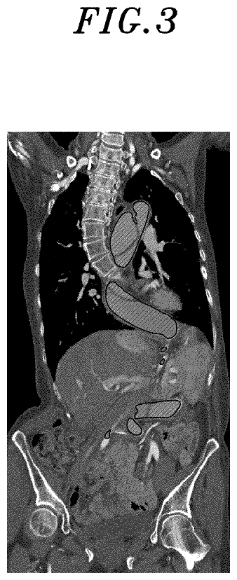

For example, the model generator 210 may create a 3D shape of blood flow based on contrasted tomographic image data of blood flow in a target body part as illustrated in FIG. 3. For example, for a vessel zone including a main vessel in biological tissues and an accessory vessel branching from the main vessel, the model generator 210 may acquire 3D blood flow shape data by performing CT imaging through Three-Dimensional Computer Tomogram Angiography (3DCTA) using a contrast agent. Alternatively, 3D blood flow shape data may be acquired through blood flow Magnetic Resonance Imaging (MRI) in which a contrast agent is injected.

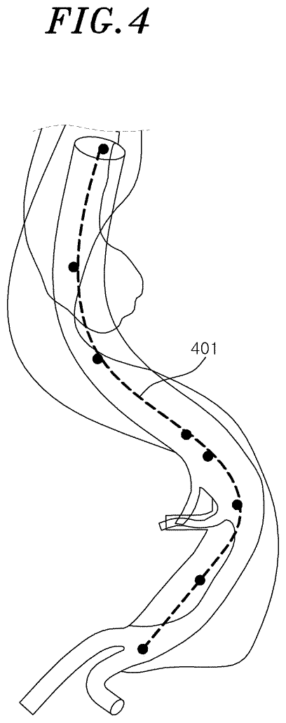

In addition, the model generator 210 may generate 3D model data, as illustrated in FIG. 4, based on 3D vascular membrane shape data acquired by adding a membrane thickness to a surface shape of the created 3D blood flow shape. For example, the model generator 210 may acquire the 3D vascular membrane model shape data corresponding to the main vessel by identifying a center line 401 for the main vessel based on the blood flow shape data and then adding, through data processing, a vascular membrane having a thickness of 24 mm to 28 mm to be symmetrical to the center line 401 on the surface of the shape data. Here, the 3D vascular membrane model corresponding to the main vessel may include the fixing hole 130 that may be used to fix the finally generated customized guide 100 to the thoracoabdominal graft. In addition, the model generator 210 may acquire 3D vascular membrane model shape data corresponding to the accessory vessel by generating a center line for the accessory vessel based on the blood flow shape data and then adding, through data processing, a vascular membrane having a thickness of 7 mm to 9 mm to be symmetrical with respect to the center line on the surface of the shape data.

Referring to FIGS. 1 and 2, the guide generator 220 generates the customized guide 100 provided with the indicator 120, which indicates the branching location and the branching direction of the accessory vessel in a body having a shape corresponding to the main vessel, based on the 3D vascular membrane model shape data acquired with respect to the biological tissues. For example, the guide generator 220 may include a 3D printer of a powder-based Color Jet Printing (CJP) or light-curable resin-based Stereo Lithography Apparatus (SLA), which creates a structure having a shape corresponding to 3D vascular membrane model shape data when the 3D vascular membrane model shape data is input. For example, the 3D printer may be a precise deposition molding machine having a jet nozzle for jetting a flexible polymer in the form of fine droplets and a jet nozzle for jetting a support material in the form of fine droplets.

The guide generator 220 may generate the customized guide 100 in a form in which the indicator 120 and the fixing hole 130 are provided in the body 110 to indicate the branching direction of the accessory vessel. For example, the guide generator 220 may include the indicator 120 that protrudes from the body 110 to have a shape corresponding to a cross section perpendicular to the blood flow direction of the accessory vessel. Here, the size of the fixing hole 130 may be determined in consideration of the size of the forceps that can be used when the finally generated customized guide 100 and the thoracoabdominal graft are fixedly coupled.

In addition, the guide generator 220 may identify the inner diameter of the main vessel based on the 3D vascular membrane model shape data, and then determine the outer diameter of the body 110 based on the inner diameter of the main vessel. For example, the customized guide 100 may be generated such that the outer diameter of the body 110 is identical to the inner diameter of the main vessel or a difference between the outer diameter of the body 110 and the inner diameter of the main vessel is smaller than a membrane thickness of the main vessel. This is because, in the case of fitting the customized guide 100 to the thoracoabdominal graft, the fixing and coupling may be easily done by sliding between the customized guide 100 and the thoracoabdominal graft when the outer diameter of the body 110 is smaller than the inner diameter of the thoracoabdominal graft.

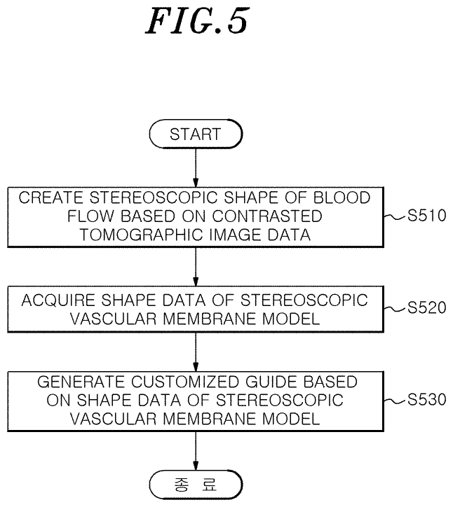

FIG. 5 is a flowchart illustrating a customized guide manufacturing method, which is performed by a customized guide manufacturing apparatus according to the embodiment of the present disclosure.

Hereinafter, a customized guide manufacturing method performed by the customized guide manufacturing apparatus 200 will be described with reference to FIGS. 1 to 5.

First, the model generator 210 of the customized guide manufacturing apparatus 200 creates a shape of a vessel based on contrasted tomographic image data (see FIG. 3) of blood flow in a target body part including a main vessel in the biological tissues and an accessory vessel branching from the main vessel. (step S510) For example, for a vessel zone including the main vessel in the biological tissues and the accessory vessel branching from the main vessel, the model generator 210 may acquire 3D blood flow shape data by performing CT imaging through Three-Dimensional Computer Tomogram Angiography (3DCTA) using a contrast agent. Alternatively, 3D blood flow shape data may be acquired through MRI of blood flow in which a contrast agent is injected.

In addition, the model generator 210 may generate 3D model data, as illustrated in FIG. 4, based on 3D vascular membrane shape data acquired by adding a membrane thickness to a surface shape of the created 3D blood flow shape.

Here, the model generator 210 may acquire the 3D vascular membrane model shape data corresponding to the main vessel by generating a center line 401 for the main vessel based on the blood flow shape data and then adding, through data processing, a vascular membrane having a thickness of 24 mm to 28 mm to be symmetrical to the center line 401 on the surface of the 3D shape data. In addition, the 3D vascular membrane model shape data for the main vessel may include the fixing hole 130 of which an inner diameter can be determined to be 2 mm to 4 mm in consideration of the size of forceps that can be used to fixedly couple the customized guide 100 and thoracoabdominal graft. In addition, the model generator 210 may acquire 3D vascular membrane model shape data corresponding to the accessory vessel in operation S520 by generating a center line for the accessory vessel based on the 3D blood flow shape data and then adding, through data processing, a vascular membrane having a thickness of 7 mm to 9 mm to be symmetrical with respect to the center line on the surface of the 3D shape data. (step S520)

Next, based on the acquired 3D vascular membrane model shape data acquired with respect to the biological tissues, the guide generator 220 generates a customized guide 100 that includes the indicator 120, which indicates a branching location and a branching direction of an accessory vessel in the body having a shape corresponding to the main vessel, and the fixing hole 130. For example, the guide generator 220 may include a 3D printer, and when the 3D vascular membrane model shape data acquired in step S520 is input, the 3D printer may generate the customized guide 100 to have a shape corresponding to the 3D vascular membrane model shape data and output the customized guide 100. (step S530)

Meanwhile, in step S530, the guide generator 220 may generate the customized guide 100 in a form in which the indicator 120 is provided in the body 110 to indicate the branching direction of an accessory vessel. For example, the guide generator 220 may include a ring-shaped indicator 120 that protrudes from the body 110 to have a shape corresponding to a cross section perpendicular to the blood flow direction of the accessory vessel. For example, the ring-shaped indicator 120 may be generated such that a side closer to the branching direction of the accessory vessel is formed to be lower than a side far from the branching direction so as to indicate the branching direction.

Further, in step S530, the guide generator 220 may identify an inner diameter of the main vessel based on the 3D vascular membrane model shape data, and then determine an outer diameter of the body 110 based on the inner diameter of the main vessel. For example, the customized guide 100 may be generated such that the outer diameter of the body 110 is the same as the inner diameter of the main vessel or a difference therebetween is smaller than a membrane thickness of the main vessel. This is because, in the case of fixing and coupling the customized guide 100 to the thoracoabdominal graft, the customized guide 100 may be easily fitted to the thoracoabdominal graft in a sliding manner and then fixed thereto through the fixing hole 130 using forceps when the outer diameter of the body 110 is smaller than the inner diameter of the thoracoabdominal graft.

According to a related art, prior to open surgery repair of thoracoabdominal aorta, a method of identifying locations of the aorta and arterioles based on a patient's medical image, and measuring an angle between the visceral artery and intercostal artery based on a cross section perpendicular to a blood flow direction of the main vessel to reconstruct a graft through mathematical calculation has been used. However, this approach has a limitation in reconstructing the graft at an accurate location because the angle is calculated by considering only the direction of arterioles.

According to the embodiment of the present disclosure, it is possible to provide a customized guide, in which the location of the accessory vessel branching from the main vessel is indicated according to the patient's anatomical structure, and also indicate the branching direction, and therefore, a location for a vascular graft to be installed in an thoracoabdominal graft and a direction of installation of the vascular graft may be guided when a graft for aortic reconstruction is manufactured.

In a method of using a customized guide according to the embodiment, it is possible to fit the customized guide to the thoracoabdominal graft, and in this case, a corresponding location of the thoracoabdominal graft is changed to a convex shape by the indicator protruding from the body of the customized guide, and thus, it is possible to guide the corresponding location as a location of the vascular graft to be installed on the thoracoabdominal graft.

Combinations of steps in each flowchart attached to the present disclosure may be executed by computer program instructions. Since the computer program instructions can be mounted on a processor of a general-purpose computer, a special purpose computer, or other programmable data processing equipment, the instructions executed by the processor of the computer or other programmable data processing equipment create a means for performing the functions described in each step of the flowchart. The computer program instructions can also be stored on a computer-usable or computer-readable recording medium which can be directed to a computer or other programmable data processing equipment to implement a function in a specific manner. Accordingly, the instructions stored on the computer-usable or computer-readable recording medium can also produce an article of manufacture containing an instruction means which performs the functions described in each step of the flowchart. The computer program instructions can also be mounted on a computer or other programmable data processing equipment. Accordingly, a series of operational steps are performed on a computer or other programmable data processing equipment to create a computer-executable process, and it is also possible for instructions to perform a computer or other programmable data processing equipment to provide steps for performing the functions described in each step of the flowchart.

In addition, each step may represent a module, a segment, or a portion of codes which contains one or more executable instructions for executing the specified logical function(s). It should also be noted that in some alternative embodiments, the functions mentioned in the steps may occur out of order. For example, two steps illustrated in succession may in fact be performed substantially simultaneously, or the steps may sometimes be performed in a reverse order depending on the corresponding function.

The above description is merely exemplary description of the technical scope of the present disclosure, and it will be understood by those skilled in the art that various changes and modifications can be made without departing from original characteristics of the present disclosure. Therefore, the embodiments disclosed in the present disclosure are intended to explain, not to limit, the technical scope of the present disclosure, and the technical scope of the present disclosure is not limited by the embodiments. The protection scope of the present disclosure should be interpreted based on the following claims and it should be appreciated that all technical scopes included within a range equivalent thereto are included in the protection scope of the present disclosure.

1. A customized guide comprising:
a body having a shape corresponding to a main vessel in biological tissues that includes the main vessel and an accessory vessel branching from the main vessel;
an indicator provided in the body to indicate a branching location of the accessory vessel.

2. The customized guide of claim 1, wherein the indicator indicates a branching direction of the accessory vessel in the body.

3. The customized guide of claim 2, wherein the indicator protrudes from the body to have a shape corresponding to a cross section perpendicular to a blood flow direction of the accessory vessel.

4. The customized guide of claim 1, wherein an outer diameter of the body is identical to an inner diameter of the main vessel or a difference between the outer diameter of the body and the inner diameter of the main vessel is smaller than a membrane thickness of the main vessel.

5. A customized guide manufacturing method performed in a customized guide manufacturing apparatus, the method comprising:
generating three-dimensional model data for a target part including a main vessel in biological tissues and an accessory vessel branching from the main vessel; and
generating a guide including an indicator that indicates a branching location of the accessory vessel in a body having a shape corresponding to the main vessel based on the three-dimensional model data.

6. The customized guide manufacturing method of claim 5, wherein in the generating the guide, the indicator indicates a direction in which the accessory vessel branches from the main vessel.

7. The customized guide manufacturing method of claim 6, wherein in the generating the guide, the indicator is provided to protrude from the body to have a shape corresponding to a cross section perpendicular to a blood flow direction of the accessory vessel.

8. The customized guide manufacturing method of claim 5, wherein in the generating the guide, the customized guide manufacturing apparatus identifies an inner diameter of the main vessel based on the three-dimensional model data and then determines an outer diameter of the body based on the inner diameter of the main vessel.

9. (canceled)

10. A custom guide manufacturing apparatus comprising:
a model generator configured to generate three-dimensional model data on a target part including a main vessel in biological tissues and an accessory vessel branching from the main vessel; and
a guide generator configured to generate a guide including an indicator that indicates a branch location of the accessory vessel on the body having a shape corresponding to the main vessel based on the three-dimensional model data.

11. The customized guide manufacturing apparatus of claim 10, wherein the indicator of the guide generator is provided to indicate a branching direction of the accessory vessel.

12. The customized guide manufacturing apparatus of claim 10, wherein the indicator of the guide generator is provided to protrude from the body to have a shape corresponding to a cross section perpendicular to a blood flow direction of the accessory vessel.

13. The customized guide manufacturing apparatus of claim 10, wherein the guide generator identifies an inner diameter of the main vessel based on the three-dimensional model data and determines an outer diameter of the body based on the inner diameter of the main vessel.
